(19) United States
(12) Patent Application Publication
(10) Pub. No.: US 2023/0418912 A1
(43) Pub. Date: Dec. 28, 2023

(54) MEDICAL DATA OWNERSHIP MANAGEMENT METHOD

(71) Applicant: ASIA UNIVERSITY, TAICHUNG CITY (TW)

(72) Inventors: ZON-YIN SHAE, TAIPEI CITY (TW); JING-PHA TSAI, TAICHUNG CITY (TW); CHI-YU CHANG, TAICHUNG CITY (TW); YUAN-YU TSAI, TAICHUNG CITY (TW); KUN-YI CHEN, TAICHUNG CITY (TW)

(21) Appl. No.: 18/046,714

(22) Filed: Oct. 14, 2022

(30) Foreign Application Priority Data
Jun. 27, 2022 (TW) 111123948

(51) Int. Cl. G06F 21/10; G16H 40/20

(52) U.S. Cl. CPC G06F 21/105; G16H 40/20

(57) ABSTRACT

A medical data ownership management method comprises: Granting a first authority to a medical unit where the first authority allows the medical unit to generate a first medical object with its corresponding first non-fungible token (NFT) storing on a blockchain. Granting a second authority to a research unit, where the second authority allows the research unit to access the first medical object, and the research unit processes the first medical object to generate the second medical object with its corresponding second NFT storing on the blockchain. Finally, granting a third authority to a user unit, where the third authority allows the user unit to access the second medical object. The access control and ownership transfer of each medial object in the blockchain are achieved by trading the corresponding NFT and modifying its subordinate list. With the management method, the security and the privacy of the medical data will be enhanced.

### CROSS-REFERENCE TO RELATED APPLICATION

This application claims the benefit of Taiwan Patent Application No. 111123948, filed on Jun. 27, 2022, in the Taiwan Intellectual Property Office, the disclosure of which is incorporated herein in its entirety by reference.

### BACKGROUND OF THE INVENTION

### 1. Field of the Invention

The present invention relates to a data management method, and more particularly, to a digital medical data ownership management method.

### 2. Description of the Related Art

The importance and value of medical data cannot be overemphasized and the medical data may have the significant potential for commercial and financial benefits in addition to application for academic research. However, in terms of privacy and security of the medical data, most of recent medical data is safely guarded and stored in databases of medical institutions, and the data may be partially explored only through multi-layered authorizations and screening processes even for academic purposes of researchers of the institutions. In addition to the authority management, when the digital type medical data involves a transfer of ownership, the situation may be much more complicated.

In brief, attributing the transfer of entity ownership is the basis of modern social financial activities such as economic activities of trading commodities. In addition, due to the popularity of computers digital data overflows all around people, when ownership of digital data is sufficiently defined or transferred, the economic activities for the purposes of business, research and innovation may be driven. However, in order to complete the concept of digital data ownership, it is necessary to clearly define the method of managing and processing data, the owner and the responsibility. When the digital data ownership cannot be defined and protected, there may be increased uncertainty about any applicable relevant agreements or contracts. In other words, even when the transfer of ownership of digital data has been completed, it is impossible to ascertain whether the original owner has resold, lent or transferred the digital data, thereby exerting a serious impact on driving the operations of the data-activated ecosystem. In terms of digital medical data, when the corresponding ownership is not completely transferred, the privacy and security of the medical data may also have a risk in addition to the impact on driving the data activation.

In conclusion, the inventor of the present invention has considered and designed the method for managing medical data ownership so as to improve the deficiencies in the related art, and increase the industrial applicability.

### SUMMARY OF THE INVENTION

For the above purposes, the present invention provides a medical data ownership management method including the following steps:
granting, by an administrator unit, a first authority to a medical unit, in which the first authority permits the medical unit to create a first medical identification object, the first medical identification object is stored on a blockchain, and the blockchain is provided with a first non-fungible token (NFT) corresponding to the first medical identification object;
granting, by the medical unit, a second authority to a research unit, in which the second authority permits the research unit to access the first medical identification object;
accessing and processing, by the research unit, the first medical identification object and accordingly creating a second medical identification object, in which the second medical identification object is stored on the blockchain, and the blockchain is provided with a second non-fungible token corresponding to the second medical identification object; and
granting, by the research unit, a third authority to a user unit, in which the third authority permits the user unit to access the second medical identification object.

The first medical identification object and the second medical identification object are associated with a first non-fungible token and a second non-fungible token, respectively. The subordinate list of the first non-fungible token records that the first medical identification object is created by the medical unit and accessed by the research unit, and the subordinate list of the second non-fungible token records that the second medical identification object is created by the research unit and accessed by the user unit.

Preferably, the first medical identification object may include medical data.

Preferably, the second medical identification object may include one of research data of the first medical identification object.

Preferably, the medical data ownership management method of the present invention further includes: withdrawing, by the administrator unit, the first authority from the medical unit.

Preferably, the medical data ownership management method of the present invention further includes: withdrawing, by the medical unit, the second authority from the research unit.

Preferably, the medical data ownership management method of the present invention further includes: withdrawing, by the research unit after permission by the medical unit, the third authority from the user unit.

Preferably, the medical data ownership management method of the present invention further includes: executing, by the research unit, a manipulation authority on the second medical identification object, in which the second medical identification object is subordinate to the first medical identification object created by the medical unit.

Preferably, the manipulation authority may include an action of selling, deriving, lending, sharing, accessing, analyzing, processing, privatizing, securing, modifying, creating, manipulating or deleting the second medical identification object, and the manipulation authority may be executed by modifying an owner of the second non-fungible token and a subordinate list thereof.

Preferably, when the research unit executes the manipulation authority and transfers the second medical identification object to other research unit, the subordinate list of the second non-fungible token may record a transfer process between the research unit and the other research unit.

Preferably, when the research unit executes the manipulation authority and transfers the second medical identification object to other research unit, the other research unit may have the third authority on the user unit, and the medical unit may have the second authority on the other research unit.

The medical data ownership management method of the present invention provides a complete method of transferring ownership of digital medical data. For a vertical medical data management, a scheme of granting and withdrawing authorizations between the administrator unit, the medical units, the research unit and the user unit, is set; and for a horizontal medical data management, the ownership management when the research unit is operated in a transverse direction is also defined. In particular, every access to the digital medical data of the present invention is performed on the blockchain, and the data cannot be forged through the blockchain itself, so that the security and accuracy of the digital medical data can be remarkably secured.

### BRIEF DESCRIPTION OF THE DRAWINGS

FIG. 1 is a flowchart of a medical data ownership management method according to the present invention.

FIG. 2 is a schematic diagram illustrating a dependency relationship between a first medical identification object and a second medical identification object according to the present invention.

FIG. 3 is a schematic diagram illustrating the medical data ownership management method according to the present invention.

FIG. 4 is a schematic diagram illustrating a transaction of the medical data ownership management method according to the present invention.

FIG. 5A is a first schematic diagram illustrating changes in authorities of the first medical identification object and the second medical identification object according to the present invention.

FIG. 5B is a second schematic diagram illustrating changes in authorities of the first medical identification object and the second medical identification object according to the present invention.

FIG. 6 is a block diagram illustrating components of a machine able to read instructions from a machine-readable medium and perform any of the methodologies discussed herein, according to an example embodiment.

### DETAILED DESCRIPTION OF THE PREFERRED EMBODIMENTS

In order to understand technical features, contents, advantages and effects implemented thereby according to the present invention, hereinafter, the present invention will be described using representative expressions of the embodiments in detail with reference to the drawings as follows. However, the drawings are only intended to supplement the specification as a gist, and may not correspond to the actual proportions and exact arrangements after implementation of the present invention. Therefore, the drawings do not limit the scope of the claims to be actually implemented by the present invention upon interpretation related to the configuration and arrangement of the accompanying drawings.

Advantages, features and technical solutions to be achieved according to the present invention will be more easily understood by describing in more detail the accompanying drawings and the embodiments as reference examples. Further, the present invention may be implemented in various forms and will not be construed as being limited only to the embodiments described herein. Whereas, for those having ordinary skill in the art, the embodiments will be provided with a more thorough understanding of the disclosure and the scope of the present invention will be fully delivered. Further, the present invention will be subject to the following claims.

According to the present invention, an administrator unit may include a system builder host, and the administrator unit may have the highest control authority. A medical unit may include a computer host representing a medical institution (such as a medical center or hospital). A research unit may include a computer host representing research teams, and the research unit is created and subordinated to the medical unit. A user unit may include a computer host representing general users. When the aforementioned units communicate with each other, each account, unique identification code, network IP, network entity location, and the like may be used to identify a unit for each type to which the unit in communication belongs.

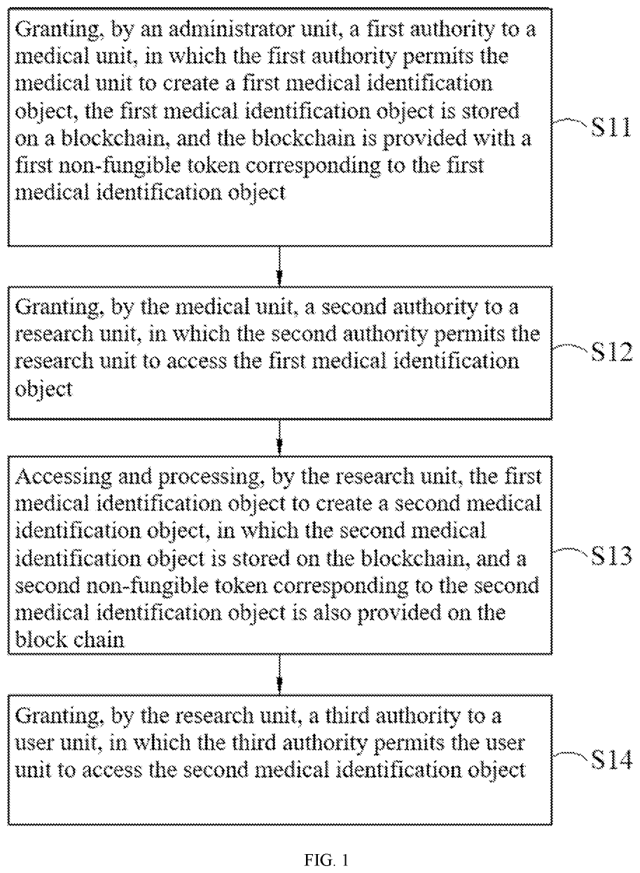

FIG. 1 is a flowchart of a medical data ownership management method according to the present invention. As shown in FIG. 1, the medical data ownership management method of the present invention includes the following steps.

In step S11, the administrator unit grants first authority A to the medical unit, and the first authority A allows the medical unit to create a first medical identification object. The first medical identification object may include medical data, such as medical history, health reports, and examination reports. The first medical identification object may be in the form of digital data and stored on the blockchain, and a first non-fungible token (NFT) corresponding to the first medical identification object is provided on the blockchain.

In step S12, the medical unit grants second authority B to the research unit, and the second authority B allows the research unit to access the first medical identification object. In particular, the research unit is subordinate to the medical unit. For example, a research organization may be subordinated to a medical institution. The medical unit may grant the second authority B to the research unit to allow the access to the first medical identification object.

In step S13, the research unit accesses and processes the first medical identification object to create a second medical identification object, in which the second medical identification object may be research data related to the first medical identification object. The second medical identification object is also stored on the block chain, and a second non-fungible token corresponding to the second medical identification object is also provided on the block chain.

In step S14, the research unit grants third authority C to the user unit, in which the third authority C allows the user unit to access the second medical identification object on the blockchain.

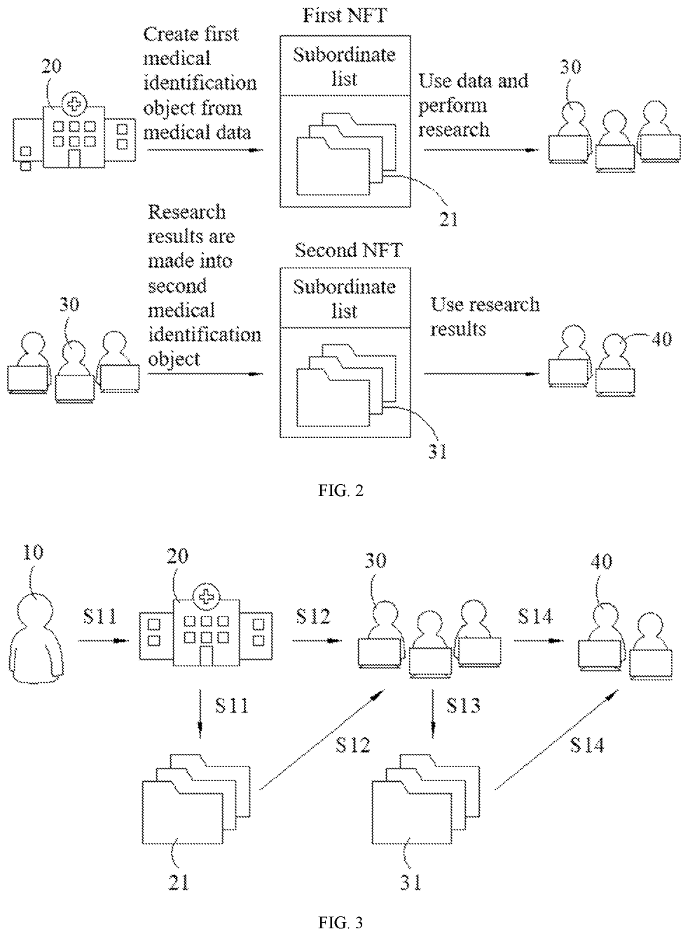

In particular, as shown in FIG. 2, the first medical identification object 21 and the second medical identification object 31 may be associated with a subordinate list (or a dependency list) of the first non-fungible token and a subordinate list (or a dependency list) of the second non-fungible token, respectively. At this point, the subordinate list of the first non-fungible token records that the first medical identification object 21 is created by the medical unit 20 and a predetermined research unit 30 performs a research access to the data. The subordinate list of the second non-fungible token records that the second medical identification object 31 is created by the research unit 30, and a predetermined user unit 40 accesses to research results. At the same time, the subordinate list may also record dependencies between corresponding medical identification objects between the non-fungible tokens. For example, the subordinate list may record that the second medical identification object 31 is created from the first medical identification object 21. Since the subordinate list of the first non-fungible token has the same structure as the subordinate list of the second non-fungible token, this embodiment is shown only with the predetermined subordinate lists. However, the present invention is not limited thereto.

In addition, the subordinate lists of the first non-fungible token and the subordinate list of the second non-fungible token may be created together when the first medical identification object 21 and the second medical identification object 31 are created, and accordingly, the subordinate lists may be stored in a form subordinated to the first non-fungible token and the second non-fungible token on the blockchain. The function of the subordinate list of the first non-fungible token and the subordinate list of the second non-fungible token is to preserve a history record when ownership of digital data is transferred. The decentralized and forgery-preventing characteristics of the blockchain are used, so that owners and accessors of the first medical identification object 21 and the second medical identification object 31 at predetermined timings are recorded in the subordinate list of the first non-fungible token and the subordinate list of the second non-fungible token.

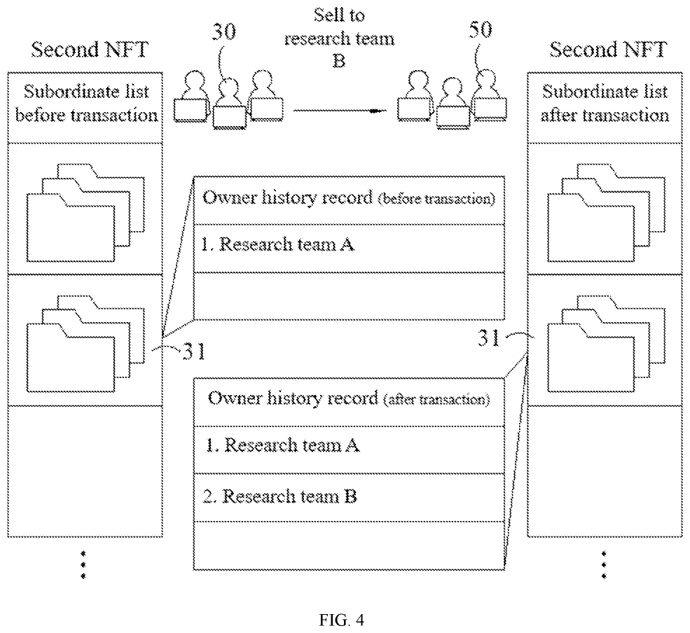

Referring to FIG. 3, it shows the process of granting authority functions according to the present embodiment. Referring to the process steps of S11 to S14 in FIG. 1 together, the figures show the authority granting process between the administrator unit 10, the medical unit 20, the research unit 30 and the user unit 40, in which the administrator unit 10 may grant the first authority A to the medical unit 20, and the first authority A may secure that the medical unit 20 is provided with the authority to create the first medical identification object 21. The medical unit 20 may grant the second authority B to the research unit 30 related thereto, in which the second authority B may secure that the research unit 30 has the authority to access the first medical identification object 21, and the research unit 30 may further create the second medical identification object 31. Finally, when the research unit 30 grants the third authority C to the user unit 40, the user unit 40 may have an authority to access the second medical identification object 31.

In addition, in one exemplary embodiment, the present invention may further include a process for withdrawing the authorities. For example, the administrator unit 10 may withdraw the first authority A from the medical unit 20, and the medical unit 20 may withdraw the second authority B from the research unit 30, or the research unit 30 after permission of the medical unit may withdraw the third authority C from the user unit 40, thereby handling the situation in which the access authority is changed when a members or an organization varies so that the method of managing ownership of medical data can be improved.

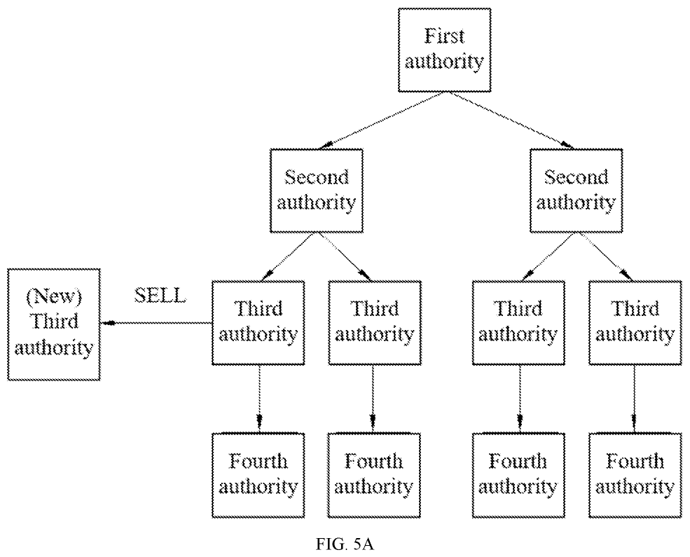

Referring to FIG. 4, this is a schematic diagram illustrating a transaction of the medical data ownership management method according to the present invention. Referring to FIGS. 1 and 3 together, as shown in the figures, in addition to the steps S11 to S14, the medical data ownership management method of the present invention may further include: executing, by the research unit a manipulation authority D on the second medical identification object 31, in which the second medical identification object 31 may be subordinate to the first medical identification object 21 created by the medical unit 20, and related records corresponding to the first medical identification object 21 and the second medical identification object 31 may be stored in the corresponding subordinate lists of the first and second non-fungible tokens on the blockchain. Besides, according to the present invention, the first medical identification object 21 and the second medical identification object 31 may support a transaction using a fungible token, which includes a fungible token such as Bitcoin or Ethereum. In addition, a range of the manipulation authority D may include an action of selling, deriving, lending, sharing, accessing, analyzing, processing, privatizing, securing, modifying, creating, manipulating or deleting the second medical identification object.

Hereinafter, sales of the manipulation authority will be described as an example in order to clearly describe the operational scheme of the present embodiment. When a research unit 30 (such as research team A) executes the manipulation authority D to sell the second medical identification object 31 to other research unit 50 (such as research team B), the subordinate list of the second non-fungible token associated with the second medical identification object 31 may record a transfer process before and after the transaction between the research team A and the research team B. When the subordinate list of the second non-fungible token is reviewed through the above scheme, it may be seen that the original owner and current owner of the second medical identification object 31, data flows, users and uses are thoroughly recorded.

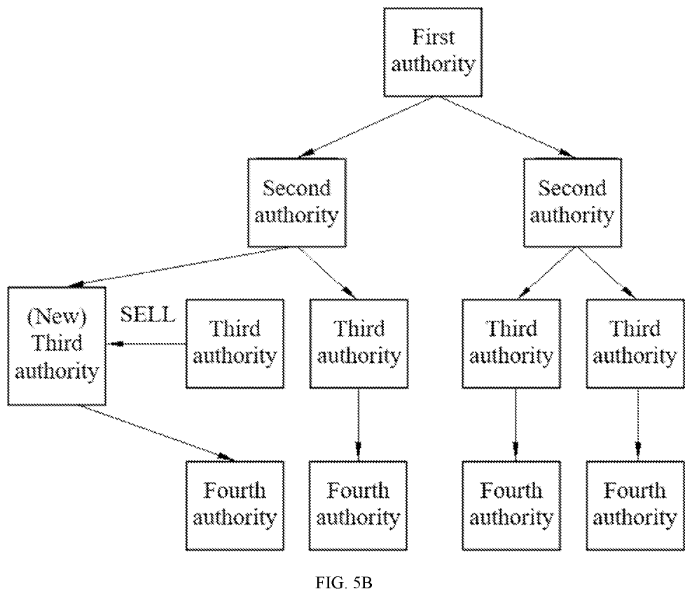

In particular, in the present embodiment, when the research team A wants to transfer the second medical identification object 31 to the research team B by executing the manipulation authority D (that is, in the case of a sale or selling transaction), the medical unit 20 originally has granted the second authority B to research team A. However the second authority B after the selling is automatically transferred to research team B, and the research team A no longer possesses the second authority B. In addition, the research team B automatically replaces and employs the same authority as the research team A. For example, the research team B has third authority C for the user unit 40, and the research team A does not have the third authority C for the user unit 40 any longer. The schematic diagrams associated changes in authority before and after the selling are shown in FIGS. 5A and 5B. In particular, the medical data is required to be carefully documented with sources, users and all outputs obtained using the medical data. Accordingly, each second medical identification object 31 may have clear transaction records and changes in authority on ownership, and the records may be retrieved from the subordinate list of the second non-fungible token. In addition, it is noted that the present embodiment has been carried out with taking the second medical identification object 31 as an example, and may be applied to the first medical identification object 21 in the same manner. Since the above manner is similar as a whole, detailed description will be omitted herein.

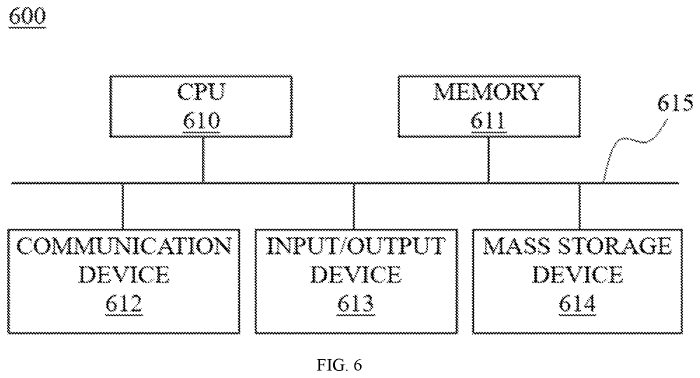

FIG. 6 is a block diagram illustrating components of a machine able to read instructions from a machine-readable medium and perform any of the methodologies discussed herein, according to an example embodiment.

The shown processing system 600 includes one or more processors, including a CPU 610, one or more memories 611 (at least a portion of which may be used as working memory, e.g., random access memory (RAM)), one or more data communication device(s) 612, one or more input/output (I/O) devices 613, and one or more mass storage devices 614, all coupled to each other through an interconnect 615. The interconnect 615 may be or include one or more conductive traces, buses, point-to-point connections, controllers, adapters and/or other conventional connection devices. Each processor 610 controls part of the operation of the processing device 600 and can be or include, for example, one or more general-purpose programmable microprocessors, digital signal processors (DSPs), mobile application processors, microcontrollers, application specific integrated circuits (ASICs), programmable gate arrays (PGAs), or the like, or a combination of such devices.

Each memory 611 can be or include one or more physical storage devices, which may be in the form of RAM, read-only memory (ROM) (which may be erasable and programmable), flash memory, miniature hard disk drive, or other suitable type of storage device, or a combination of such devices. Each mass storage device 614 can be or include one or more hard drives, digital versatile disks (DVDs), flash memories, or the like. Each memory 611 and/or mass storage 614 can store (individually or collectively) data and instructions that configure the processor(s) 610 to execute operations to implement the techniques described above. Each communication device 612 may be or include, for example, an Ethernet adapter, cable modem, Wi-Fi adapter, cellular transceiver, baseband processor, Bluetooth or Bluetooth Low Energy (BLE) transceiver, or the like, or a combination thereof. Depending on the specific nature and purpose of the processing system 600, each I/O device 613 can be or include a device such as a display (which may include a transparent AR display surface), audio speaker, keyboard, mouse or other pointing device, microphone, camera, etc. Note, however, that such I/O devices may be unnecessary if the processing device 600 is embodied solely as a server computer.

In the case of a user device, a communication device 612 can be or include, for example, a cellular telecommunications transceiver (e.g., 3G, LTE/4G, 5G), Wi-Fi transceiver, baseband processor, Bluetooth or BLE transceiver, or the like, or a combination thereof. In the case of a server, a communication device 612 can be or include, for example, any of the aforementioned types of communication devices, a wired Ethernet adapter, cable modem, DSL modem, or the like, or a combination of such devices.

Unless contrary to physical possibility, it is envisioned that (i) the methods/operations described herein may be performed in any sequence and/or in any combination, and that (ii) the components of respective embodiments may be combined in any manner.

The machine-implemented operations described above can be implemented by programmable circuitry programmed/configured by software and/or firmware, or entirely by special-purpose (“hardwired”) circuitry, or by a combination of such forms. Such special-purpose circuitry (if any) can be in the form of, for example, one or more application-specific integrated circuits (ASICs), programmable logic devices (PLDs), field-programmable gate arrays (FPGAs), system-on-a-chip systems (SOCs), etc.

Software or firmware to implement the techniques introduced here may be stored on a computer-readable storage medium and may be executed by one or more general-purpose or special-purpose programmable microprocessors. A “computer-readable medium”, as the term is used herein, includes any mechanism that can tangibly store information in a non-transitory form accessible by a machine (a machine may be, for example, a computer, network device, cellular phone, personal digital assistant (PDA), manufacturing tool, any device with one or more processors, etc.). For example, a computer-readable medium includes recordable/non-recordable media (e.g., RAM or ROM; magnetic disk storage media; optical storage media; flash memory devices; etc.), etc.

As can be seen from the above description, the medical data ownership management method of the present invention can elevate the use of medical data of each medical institution, clearly define the method of managing and processing the digital data ownership, and enable data owner to truly own and pro-actively control the authority on the data of the owner to freely decide using scheme on the digital data in various situations. The medical data is set on the blockchain, so that ownership on the medical data is proved through the subordinate list of the corresponding non-fungible token. Academic and commercial values, such as transaction, rental and sale of the ownership, of the medical data are activated. The above concept may be extended and applied to various fields in addition to the medical field, so that the design management for the ownership over digital data created in the various fields may be provided.

The above-described embodiments have merely described the technical spirit and features of the present invention. The purpose is only for those skilled in the art to understand the content and implementation of the present invention, and the description herein is not intended to limit the scope of the present invention. Therefore, modifications and improvements according to the ideas disclosed in the present invention are still within the scope of the present invention.

What is claimed is:

1. A medical data ownership management method comprising:
granting, by an administrator unit, a first authority to a medical unit, in which the first authority permits the medical unit to create a first medical identification object, the first medical identification object is stored on a blockchain, and the blockchain is provided with a first non-fungible token corresponding to the first medical identification object;
granting, by the medical unit, a second authority to a research unit, in which the second authority permits the research unit to access the first medical identification object;
accessing and processing, by the research unit, the first medical identification object to create a second medical identification object, in which the second medical identification object is stored on the blockchain, and a second non-fungible token corresponding to the second medical identification object is also provided on the block chain; and
granting, by the research unit, a third authority to a user unit, in which the third authority permits the user unit to access the second medical identification object, wherein
the first medical identification object and the second medical identification object are associated with a subordinate list of the first non-fungible token and a subordinate list of the second non-fungible token, respectively, in which the subordinate list of the first non-fungible token records that the first medical identification object is created by the medical unit and accessed by the research unit, and the subordinate list of the second non-fungible token records that the second medical identification object is created by the research unit and accessed by the user unit.

2. The medical data ownership management method of claim 1, wherein the first medical identification object includes medical data.

3. The medical data ownership management method of claim 1, wherein the second medical identification object includes one of research data of the first medical identification object.

4. The medical data ownership management method of claim 1, further comprising:
withdrawing, by the administrator unit, the first authority from the medical unit.

5. The medical data ownership management method of claim 1, further comprising:
withdrawing, by the medical unit, the second authority from the research unit.

6. The medical data ownership management method of claim 1, further comprising:
withdrawing, by the research unit after permission of the medical unit, the third authority from the user unit.

7. The medical data ownership management method of claim 1, further comprising:
executing, by the research unit, a manipulation authority on the second medical identification object, in which the second medical identification object is subordinate to the first medical identification object created by the medical unit.

8. The medical data ownership management method of claim 7, wherein the manipulation authority includes an action of selling, deriving, lending, sharing, accessing, analyzing, processing, privatizing, securing, modifying, creating, manipulating or deleting the second medical identification object, and the manipulation authority is executed by modifying an owner of the second non-fungible token and a subordinate list thereof.

9. The medical data ownership management method of claim 7, wherein, when the research unit executes the manipulation authority and transfers the second medical identification object to one other research unit, the subordinate list of the second non-fungible token records a transfer process between the research unit and the other research unit.

10. The medical data ownership management method of claim 7, wherein, when the research unit executes the manipulation authority and transfers the second medical identification object to one other research unit, the other research unit has the third authority on the user unit, and the medical unit has the second authority on the other research unit.
